(19) United States
(12) Patent Application Publication
(10) Pub. No.: US 2005/0080441 A1
(43) Pub. Date: Apr. 14, 2005

(54) Surgical instruments which are especially useful for ophthalmic surgical procedures, and methods of making the same

(75) Inventors: Brian C. Dodge, Hillsborough, NC (US); Ronald F. Overaker, Durham, NC (US); Richard B. Nappi, Durham, NC (US)

Correspondence Address: NIXON & VANDERHYE, PC, 1100 N GLEBE ROAD, 8TH FLOOR, ARLINGTON, VA, 22201-4714, US

(73) Assignee: Duke University, Durham, NC (US)

(21) Appl. No.: 10/682,835

(22) Filed: Oct. 10, 2003

(51) Int. Cl. A61B 17/32

(52) U.S. Cl. 606/171; 606/167

(57) ABSTRACT

Surgical instrument include a longitudinally sectioned needle member establishing respective needle segments, and an outer sleeve member which exerts a circumferentially radial compression onto the needle segments yet allows for relative longitudinal movements between at least one and another of the needle segments. The needle member may be longitudinally bifurcated so as to establish said one and another needle segments. Alternatively, the needle member may be longitudinally trifurcated so as to establish a pair of stationary outer needle segments, and an intermediate needle segment sandwiched between but longitudinally moveable relative to said outer needle segments. Thus, collectively the needle segments define respective opposed longitudinal bearing edges which contact one another and thereby allow for one of the needle segments to be moveable longitudinally relative to another of the needle segments. As such, the lumen within the sectioned needle member may be maximized while the overall dimension of the instrument is minimized.

### FIELD OF THE INVENTION

The present invention relates generally to surgical implements and techniques, especially ophthalmic microsurgical instruments.

### BACKGROUND OF THE INVENTION

During ophthalmic microsurgery, such as lens removal, instruments are used to cut and remove unwanted tissues, vitreous and the like. There are, of course, numerous examples of ophthalmic surgical instruments which are adapted to remove vitreous and other tissue during an ophthalmic surgical procedure, as shown in U.S. Pat. No. 3,776,238 to Peyman et al and U.S. Pat. No. 4,986,827 to Akkas et al (the entire content of each being expressly incorporated hereinto by reference). However, each of these prior examples of conventional ophthalmic surgical instruments rely on a coaxially sleeved “tube-in-tube” needle structure whereby an inner tube is moveable relative to a stationary outer tube so as to perform cutting and aspiration. As may be appreciated, as the size of the needle tubes deceases, the effective lumen diameter will also decrease thereby potentially resulting in less than desirable aspiration through the needle tubes of unwanted biological material from the surgical site. This decrease in lumen size is therefore exacerbated when there is a need for a pair of coaxially sleeved needle tubes as in the prior art. Thus, the effective size of the instrument may be limited somewhat by the countervailing need to have sufficient lumen diameter to allow for adequate aspiration of unwanted biological material from the surgical site.

According to the present invention, however, the further miniaturization of ophthalmic microsurgical instruments is enabled largely due to the fact that the conventional need for a “tube-in-tube” cutter assembly may now be avoided.

Broadly, therefore, the present invention is embodied in microsurgical instruments whereby a needle member is longitudinally sectioned to form respective needle segments, and a sleeve member surrounding the needle segments exerting circumferential radial compressive force thereon. Thus, collectively the needle segments define respective opposed longitudinal bearing edges which contact one another and thereby allow for one of the needle segments to be moveable longitudinally relative to another of the needle segments. As such, the lumen within the sectioned needle member may be maximized while the overall dimension of the instrument is minimized.

In one preferred embodiment of the present invention, the needle member may be longitudinally bifurcated so as to establish said one and another needle segments. Alternatively, according to another preferred embodiment of the present invention, the needle member may be longitudinally trifurcated so as to establish a pair of stationary outer needle segments, and an intermediate needle segment sandwiched between but longitudinally moveable relative to said outer needle segments.

These and other aspects and advantages will become more apparent after careful consideration is given to the following detailed description of the preferred exemplary embodiments thereof.

### BRIEF DESCRIPTION OF THE ACCOMPANYING DRAWINGS

Reference will hereinafter be made to the accompanying drawings, wherein like reference numerals throughout the various FIGURES denote like structural elements, and wherein;

FIG. 1 is a perspective view of a surgical instrument which includes one preferred embodiment of a microsurgical needle blade assembly in accordance with the present invention;

FIG. 2 is an enlarged perspective view of the distal end of the microsurgical instrument depicted in FIG. 1;

FIG. 3 is an enlarged perspective view showing another embodiment of a microsurgical needle blade assembly in accordance with the present invention.

### DETAILED DESCRIPTION OF THE INVENTION

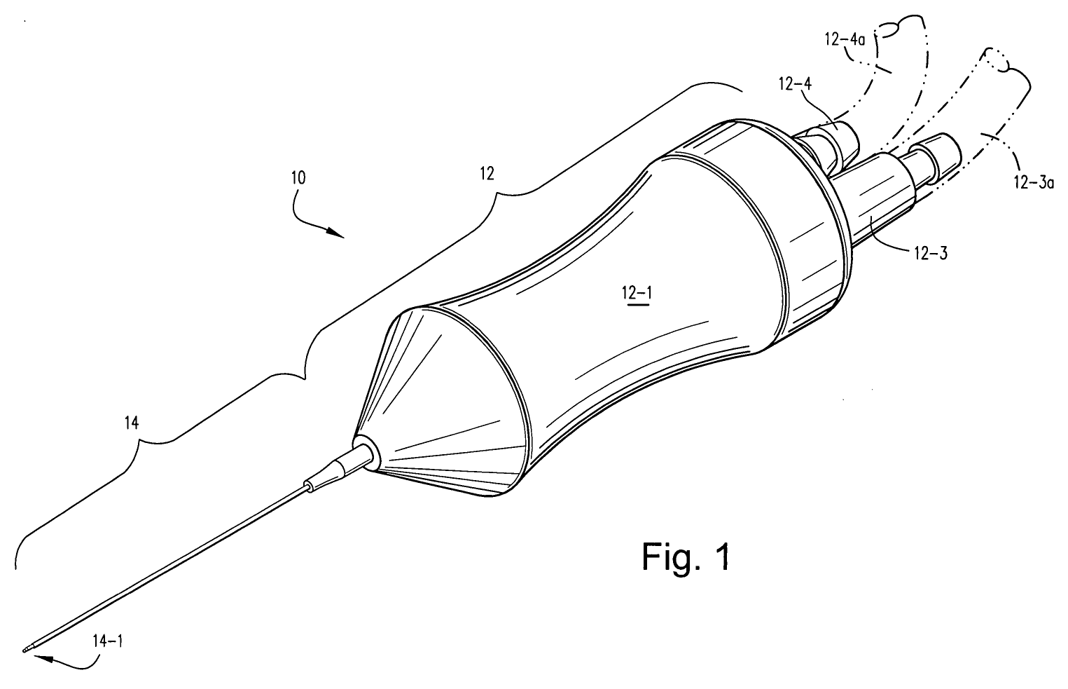

One preferred embodiment of a surgical instrument 10 according o the present invention is depicted in accompanying FIG. 1. As shown, the instrument 10 includes a combined handle/drive assembly 12, and a needle cutter blade assembly 14 operatively coupled to the handle/drive assembly 12 and distally extending therefrom. Virtually any conventional handle/drive assembly 12 may be provided in accordance with the present invention, such as those described more fully in the above-cited U.S. Pat. Nos. 3,776,238 and 4,986,827. One particularly preferred handle/drive assembly that may be incorporated for use with the needle cutter blade assembly 14 to be described in grater detail below is the ACCURUS® Surgical System commercially available from Alcon Laboratories. In generally, the handle/drive assembly 12 will most preferably include a handle member 12-1 which is sized and configured so as to be manually manipulated by an attending surgeon and which houses the drive components (not shown) of the system. Such drive components are most preferably activated by means of pneumatic pressure pulses via port 12-3 connected to a control source (not shown) thereof via tubing 12-3a. An aspiration port 12-4 may likewise be connected to a vacuum source (not shown) via tubing 124a to allow vitreous and/or tissue removed from the surgical site to be aspirated externally of the patient.

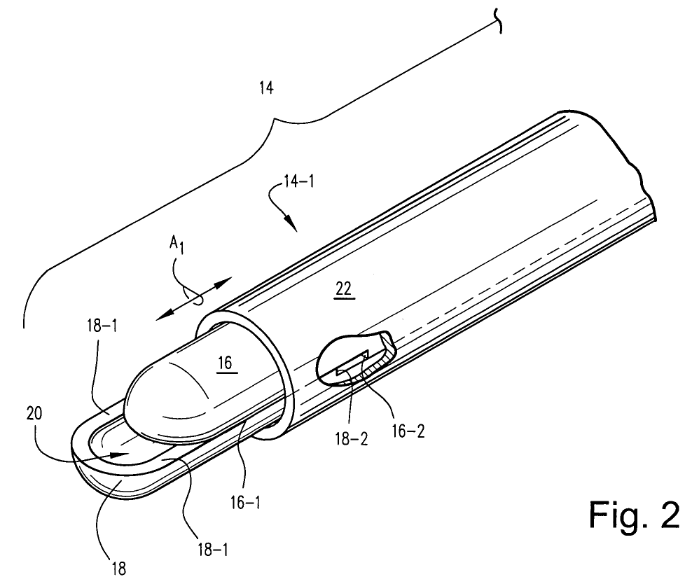

Accompanying FIG. 2 shows in an enlarged manner the distal end 14-1 of the cutter blade assembly 14. As is seen, the cutter blade assembly 14 is formed by a longitudinally bifurcated needle which establish a pair of cooperating needle segments 16, 18. The needle segments 16, 18 thus establish opposed pairs of longitudinally extending edges 16-1, 18-1, respectively, which bear against one another to allow one of the segments 16 or 18 to slide reciprocally against and relative to the other of the segments 16 or 18. Collectively, therefore, the needle segments 16, 18 define the entirety of the internal lumen 20 of the cutter blade assembly 14.

As shown in FIG. 2, the needle segment 16 is moveable while the needle segment 18 is stationary. Thus, the proximal end of the needle segment 16 includes an integral drive attachment piece (not shown) which operatively cooperates with the particular pneumatic drive mechanism employed as part of the handle/drive assembly 12 to thereby cause the segment to move reciprocally (arrow A1) along the longitudinal axis of the cutter assembly 14. Conversely, the needle segment 18 may have a proximal attachment piece (not shown) which allows it to be positionally fixed (immovable) relative to the handle/drive assembly 12. Of course, the particular hand/drive assembly 12 will dictate the particular configurations of the attachment pieces associated with the distal ends of the needle segments 16, 18, the specific design of which is well within the skill of those in this art.

The relative positions and movements of the segments 16, 18 are achieved by means of a tubular sleeve 22. The sleeve 22 is dimensioned in such a manner that sufficient circumferential radial compression of the needle segments 16, 18 occurs so as to maintain the relative positions of the segments 16, 18, while yet allowing for relative longitudinal movements to occur between the opposed contacting bearing edges 16-1, 18-1. Advancement of the distalmost tip of segment 16 beyond the distalmost tip of segment 18 may be prevented by means of notched stops 16-2, 18-2 formed in each segment proximally of their tips.

Although the needle cutter assembly 14 is depicted in accompanying FIG. 2 as being longitudinally bifurcated into substantially symmetrical needle segments 16, 18, it is within the scope of this invention that asymmetric bifurcation could be provided and in some cases may be desirable. Thus, an asymmetric longitudinal bifurcation would allow for one of the segments 16, 18 to be larger and stationary relative to the other of the smaller, moveable segments 16, 18. This larger stationary segment would therefore allow for a greater amount of vitreous and/or tissue to be received therein which could then be severed via movement of the moveable segment. Conversely, of course, the larger segment may be moveable and the smaller segment stationary.

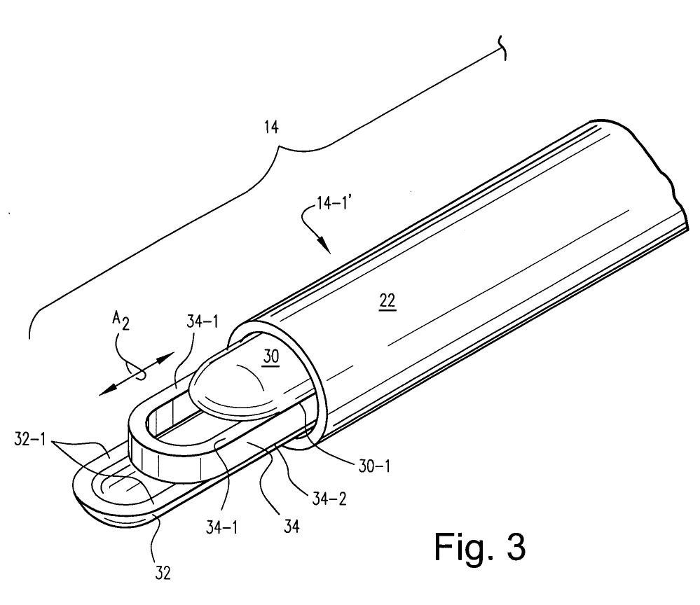

Accompanying FIG. 3 depicts another preferred exemplary embodiment of a cutter assembly 14′ that may be operatively connected to the handle/drive assembly 12. In this regard, the cutter assembly 14′ is in the form of a microneedle that has been trifurcated to establish opposed stationary (immovable) needle segments 30, 32 and a generally hairpin-shaped (U-shaped) intermediate segment 34 moveably sandwiched therebetween. Thus, the segments 30, 32 each define edges 30-1, 32-1 which bear against the respective bearing surfaces 34-1, 34-2 of the intermediate segment 34 as it moves longitudinally (arrow A2) relative to the longitudinal axis of the cutter assembly 14. As in the embodiment depicted in FIG. 2, movements of the intermediate segment 34 is achieved by means of a tubular sleeve 22 exerting sufficient radial compression onto the segments 30, 32 so that they maintain their relative positions while yet allowing for relative longitudinal movements to occur between the opposed contacting bearing edges 30-1 and 34-1 on the one hand, and 32-1 and 34-2 on the other hand. Although not shown in FIG. 2, the needle segments 30, 32 and/or 34 may also be provided with a stop similar to the stops 16-2, 18-2 depicted in FIG. 2 so to limit the extent of longitudinal movement thereof.

The needle segments are most preferably formed by any conventional micromachining technique. By way of example, a microneedle, preferably about 20 gauge or smaller (e.g., between about 20 gauge to about 25 gauge or smaller) may be longitudinally bifurcated to form segments 16, 18 by means of conventional electrical discharge machining (EDM) techniques. Thus, the distal end of a length of microtubing (e.g., a section of a conventional 20 ga. or smaller stainless steel tubular needle) may be axially translated relative to an EDM wire provided as a component part of a conventional EDM system. When energized, the EDM wire thereby bifurcates the needle as it is translated longitudinally relative to the wire. Once the needle has been bifurcated, the distalmost end may be formed into a smooth tip by reforming, machining and/or soldering.

The outer sleeve 22 is most preferably formed of a plastics material. Plastics materials which have cold-flowable properties are especially presently preferred. By cold-flowable property is meant the permanent distortion, deformation of dimensional change which occurs in a plastics material under continuous load at temperatures below the heat-softening temperature. One preferred cold-flowable plastics material is polyimide. Pre-formed polyimide tubing is commercially available from Microtubing, Inc. of Tampa, Fla. and may be satisfactorily employed in the practice of the present invention. In such a situation, the needle segments will be positioned within the preformed tubing forming the sleeve 22.

Thermoplastic materials may also be employed for use in the outer sleeve 22 according to the present invention and may be extruded onto the needle segments 16, 18. Especially preferred thermoplastic materials include polymethylpentenes (e.g., TPX® plastics commercially available from Mitsui Products) and polyacetals (e.g., DELRIN® polyacetal commercially available from DuPont). IN such a situation, the thermoplastic material may be extruded onto an exterior circumferential surface of the needle segments thereby forming the sleeve 22.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

1. A surgical instrument comprising a longitudinally sectioned needle member establishing respective needle segments, and an outer sleeve member which exerts a circumferentially radial compression onto said needle segments yet allows for relative longitudinal movements between at least one and another of said needle segments.

2. The surgical instrument of claim 1, wherein said needle member is longitudinally bifurcated so as to establish said one and another needle segments.

3. The surgical instrument of claim 1, wherein said needle member is longitudinally trifurcated so as to establish a pair of stationary outer needle segments, and an intermediate needle segment sandwiched between but longitudinally moveable relative to said outer needle segments.

4. The surgical instrument of claim 2 or 3, wherein said outer sleeve member is formed of a plastics material.

5. The surgical instrument of claim 4, wherein said outer sleeve member is formed of a plastics material at least one selected from the group consisting of polyimides, polyacetals and polymethylpentenes.

6. The surgical instrument of claim 1, further comprising a handle connected operatively to a proximal end of said longitudinally sectioned needle member.

7. The surgical instrument of claim 6, wherein said one needle segment is positionally fixed to said handle, and wherein said another needle segment is coupled operatively to said handle to allow for relative reciprocal longitudinal movements thereof.

8. The surgical instrument of claim 6, wherein said needle member is longitudinally trifurcated so as to establish a pair of outer needle segments which are immovably fixed to said handle, and an intermediate needle segment sandwiched between said pair of outer needle segments, said intermediate needle segment being connected operatively to said handle for reciprocal longitudinal movements relative to said outer needle segments.

9. The surgical instrument of claim 7 or 8, wherein said outer sleeve member is formed of a plastics material.

10. The surgical instrument of claim 9, wherein said outer sleeve member is formed of a plastics material at least one selected from the group consisting of polyimides, polyacetals and polymethylpentenes.

11. The surgical instrument of claim 1, 2 or 3 which further comprises forming a stop in at least one of said needle segments to limit an extent of longitudinal movement thereof.

12. A method of making a surgical instrument comprising:
(i) longitudinally sectioning an elongate needle member to thereby establish respective needle segments, and
(ii) positioning the needle segments within an outer sleeve member and allowing the sleeve member to circumferentially radially compress the needle segments yet allow for relative longitudinal movements between at least one and another of the needle segments.

13. The method of claim 12, wherein step (i) includes longitudinally bifurcating said needle member so as to establish said one and another needle segments.

14. The method of claim 12, wherein step (i) includes longitudinally trifurcating said needle member so as to establish a pair of stationary outer needle segments, and an intermediate needle segment sandwiched between but longitudinally moveable relative to said outer needle segments.

15. The method of claim 13 or 14, wherein step (ii) includes forming said outer sleeve of a plastics material.

16. The method of claim 15, wherein the sleeved is formed of a plastics material at least one selected from the group consisting of polyimides, polyacetals and polymethylpentenes.

17. The method of claim 12, wherein step (ii) includes positioning the needle segments within a preformed plastics tube.

18. The method of claim 12, wherein step (ii) includes extruding a thermoplastics material onto an exterior surface of the longitudinally sectioned needle member.

19. The method of claim 12, which further comprises joining said longitudinally sectioned needle member to a handle.

20. The method of claim 12, wherein step (i) is practiced using electrical discharge machining (EDM).
